(19) United States
(12) Patent Application Publication
(10) Pub. No.: US 2013/0178874 A1
(43) Pub. Date: Jul. 11, 2013

(54) COMPOSITE IMPLANT

(71) Applicant: Hilton Becker, Boca Raton, FL (US)

(72) Inventor: Hilton Becker, Boca Raton, FL (US)

(21) Appl. No.: 13/549,623

(22) Filed: Jul. 16, 2012

Related U.S. Application Data
(63) Continuation-in-part of application No. 13/534,441, filed on Jun. 27, 2012.
(60) Provisional application No. 61/509,369, filed on Jul. 19, 2011.

(51) Int. Cl. A61F 2/00

(52) U.S. Cl. CPC A61F 2/0063
USPC 606/151

(57) ABSTRACT

A composite implant includes a mesh scaffold having a biologically-active material configured to contact biological tissue and blood vessels; a resin disposed on the mesh scaffold; and a channel in the mesh which is configured to receive growth of the biological tissue and blood vessels, wherein the resin is biocompatible and non-absorbable. A process for preparing the composite implant includes disposing a plurality of layers of biologically-active material as an array; contacting the plurality of layers with a resin; and hardening the resin to form the composite implant. A process of using the composite implant includes implanting the composite implant into a subject, wherein the implant comprises: a mesh comprising a biologically-active material configured to contact biological tissue and blood vessels; a resin disposed on the mesh; and a channel in the mesh which is configured to receive growth of the biological tissue and blood vessels.

### CROSS REFERENCE TO RELATED APPLICATION

This application is a continuation-in-part of U.S. application Ser. No. 13/534,441 filed Jun. 27, 2012, which claims the benefit of U.S. Provisional Application Ser. No. 61/509,369 filed Jul. 19, 2011, the entire disclosure of each of which is hereby incorporated by reference in its entirety.

### BACKGROUND

An implant may be introduced into a human body to replace, support, or enhance a structure within the body. When a foreign body is introduced into a human body as an implant, it may be encapsulated by scar tissue, forming a capsule. Scar tissue includes the protein collagen, which in scar tissue may be cross-linked and aligned in a single direction. This may cause scar tissue to have relatively lower functional quality than collagen in normal, non-scar tissue. Thus, an implant surrounded by a scar tissue capsule may not be well integrated to the rest of the biological structures within the body, and have an undesirably low level of bio-integration.

There have been various attempts to improve bio-integration of implants. Surface texturing of an implant made of silicone creates a porous, sponge-like surface. Living body tissue may grow into the cavities to fix the implant to the body. However, a living body may react to synthetic material such as silicone by forming a capsule of scar tissue around it (as an oyster forms a pearl around a grain of sand). A non-living tissue implanted in the human body that becomes encapsulated with scar tissue may have several detrimental effects, including bone erosion. Also, if a non-living tissue is exposed through the skin, it may become infected.

Also, materials such as hyaluronic acid, collagen, and polylactic acid may be applied to the surface of an implant. Living tissue will grow into these biologically-active materials, encouraging bio-integration of the implant in the body. However, these small, biologically-active materials may be absorbed into the blood supply within living tissue that grows near the implant.

### BRIEF DESCRIPTION

The above and other deficiencies of the prior art are overcome by, in an embodiment, a composite implant comprising: a mesh comprising a biologically-active material configured to contact biological tissue and blood vessels; a resin disposed on the mesh; and a channel in the mesh which is configured to receive growth of the biological tissue and blood vessels, wherein the resin is biocompatible and non-absorbable.

In another embodiment, a process for preparing a composite implant comprises: disposing a plurality of layers of biologically-active material as an array; contacting the plurality of layers with a resin; and hardening the resin to form the composite implant, wherein a channel in the mesh is configured to receive growth of the biological tissue and blood vessels, and the resin is biocompatible and non-absorbable.

In a further embodiment, a process of using a composite implant comprises: implanting the composite implant into a subject, wherein the implant comprises: a mesh comprising a biologically-active material configured to contact biological tissue and blood vessels; a resin disposed on the mesh; and a channel in the mesh which is configured to receive growth of the biological tissue and blood vessels, wherein the resin is biocompatible and non-absorbable.

It is to be understood that both the foregoing general description and the following detailed description are exemplary and explanatory and are intended to provide further explanation of the invention as claimed.

### BRIEF DESCRIPTION OF THE DRAWINGS

The following descriptions should not be considered limiting in any way. With reference to the accompanying drawings, like elements are numbered alike:

FIG. 1 is an image of a hair;

FIG. 2 is a cross-sectional view of the hair shown in FIG. 1;

FIG. 3 shows a cross-sectional view of an implant;

FIG. 4 shows the implant of FIG. 3 after being implanted in a living body;

FIG. 5 shows a cross-sectional view of a surface-textured implant;

FIG. 6 shows the surface-textured implant of FIG. 5 after being implanted in a living body;

FIG. 7 shows a cross-sectional view of a surface-textured implant including a biologically-active matrix material;

FIG. 8 shows the surface-textured implant including a biologically-active matrix material of FIG. 7 after being implanted in a living body;

FIG. 9 shows a cross-sectional view of a surface-textured implant including a biologically-active matrix material;

FIG. 10 shows the surface-textured implant including a biologically-active matrix material of FIG. 9 after being implanted in a living body;

FIG. 11 shows a cross-sectional view of an implant with strands of biologically-active matrix material disposed therein according to an exemplary embodiment;

FIG. 12 shows a bottom view of the implant shown in FIG. 11;

FIG. 13 shows a top view of the implant shown in FIG. 11;

FIG. 14 and FIG. 15 show a cross-sectional view of an implant in accordance with an exemplary embodiment and an inset view, respectively;

FIG. 16 shows a cross-sectional view of an implant with a biologically-active matrix material according to an exemplary embodiment;

FIG. 17 shows a cross-sectional view of an implant with granules of biologically-active matrix material according to an exemplary embodiment; and

FIG. 18 shows a perspective view of a composite implant having a biologically-active material disposed in resin;

FIG. 19 shows a perspective view of the composite implant of FIG. 18 after absorption of the biologically-active material;

FIG. 20 shows a perspective view of a configuration of biologically-active material according to an embodiment;

FIG. 21 shows a perspective view of a configuration of biologically-active material having a partially interwoven structure according to an embodiment;

FIG. 22 shows a perspective view of a configuration of biologically-active material having a completely interwoven structure according to an embodiment;

FIG. 23 shows a cross-section of a composite implant with a biologically-active mesh disposed as layers in a resin;

FIG. 24 shows a cross-section of the composite implant of FIG. 23 without biologically-active mesh;

FIG. 25 shows growth of blood vessels and biological tissue into the biologically-active mesh of the composite implant of FIG. 23;

FIG. 26 shows a cross-section of a composite with growth of blood vessels and biological tissue in a channel formed in a biologically-active material disposed in a resin;

FIG. 27 shows a composite implant with resin disposed on the surface of a biologically-active mesh;

FIG. 28 shows a composite implant with stacked biological mesh layers of biologically-active material coated with a resin as in FIG. 23; and

FIG. 29 shows a chin implant made of a composite implant and an enlarged view of growth of blood vessels and biological tissue in a biologically-active material of the composite implant having a structure similar to that shown in FIG. 18.

### DETAILED DESCRIPTION

A detailed description of one or more embodiments is presented herein by way of exemplification and not limitation with reference to the figures.

Human teeth, nails, and hair have similar structures with each other, in that they are formed from living tissue within the body, and then through a transitional structure become non-living but remain integrally attached to the living tissue of the body. This property improves bio-integration and decreases the ability of teeth, nails, and hair to detach from the body. The base of these structures is living vascularized cellular (i.e., biological) material, while the distal end is non-living, non-vascularized acellular material.

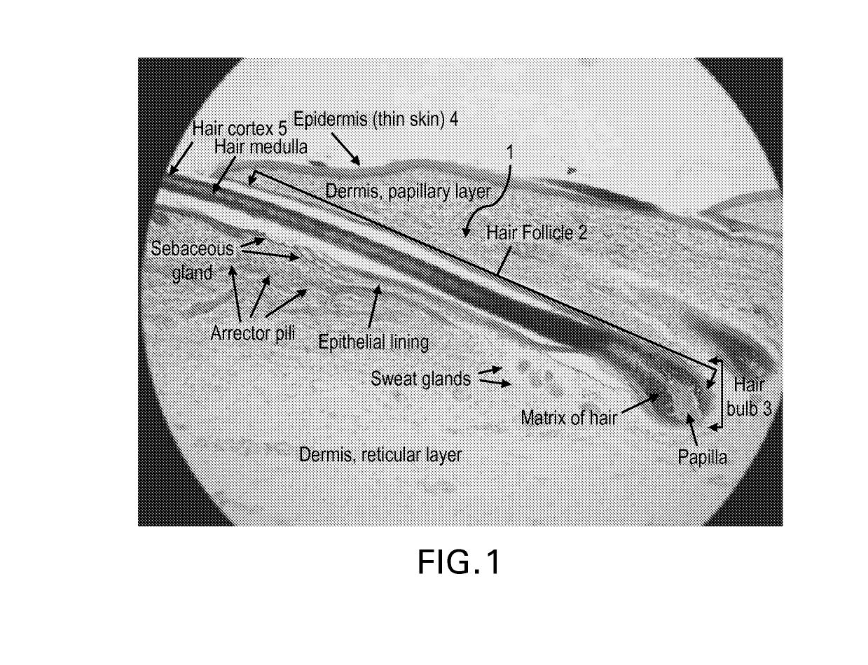

FIG. 1 shows an image of a hair 1. A follicle 2 and hair bulb 3 are regions beneath the skin 4 and within the body which grow the hair 1. The follicle 2 is the living portion of the hair 1, and the hair bulb 3 contains cells that produce a hair shaft 5. The end of the follicle 2 and the hair bulb 3 show an area where living tissue and blood supply connect to the hair 1. The hair shaft 5 is the visible portion of the hair 1 that extends beyond the skin 4 and body. The hair shaft 5 exhibits no biochemical activity and is considered non-living. A portion of the hair 1 attached to living tissue through the transitional structure may not become infected although it is exposed outside the body.

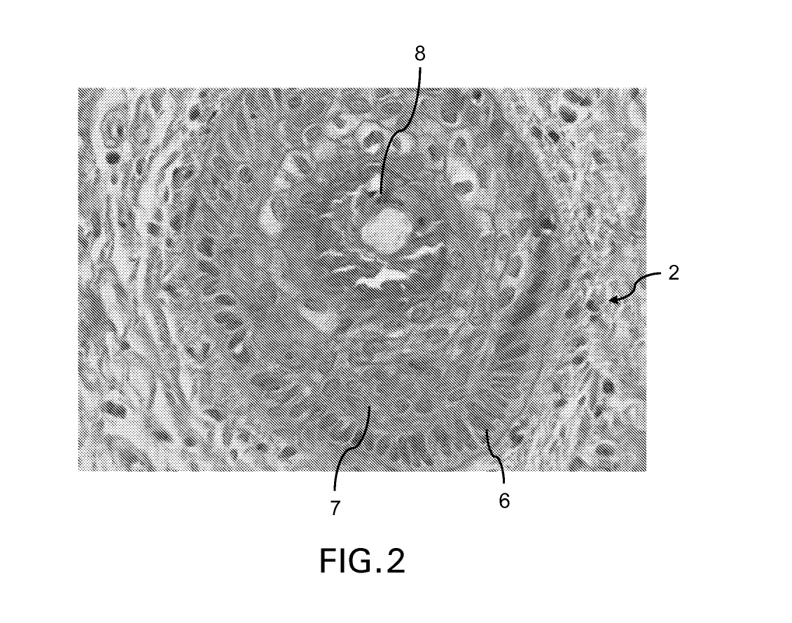

FIG. 2 shows a cross-sectional view of the hair follicle 2. The outer-most layer of hair is the cuticle 6, which is several layers of flat, thin living cells that rely on a blood supply The next inner layer is the cortex 7, which is a transitional zone that comprises cells that survive on tissue fluid only and contain protein in rod-like structures. The inner-most layer is the medulla 8, which is a disorganized area of cells at the follicle's center. In this area (medulla 8) the cells are no longer viable and are forming the hair shaft.

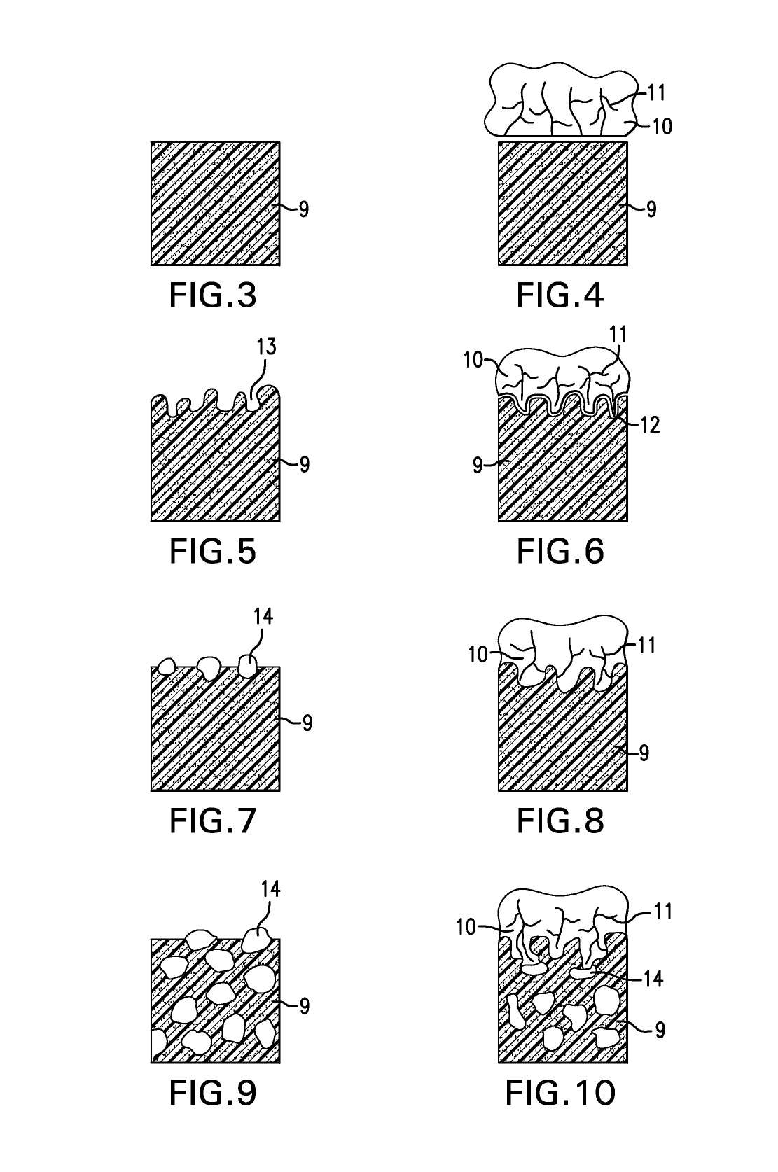

FIG. 3 through FIG. 10 shows various cross-sectional views of examples of implants and subsequent bio-integration of the implants. For simplicity, only bio-integration of the top surface of each implant is shown. However, bio-integration may occur at other surfaces of the implant. Specifically, FIG. 3 shows an implant 9, and FIG. 4 shows the implant 9 after being implanted in a living body and encapsulation has occurred. Living tissue 10 and blood vessels 11 grow near the surface of the implant 9; however, a scar tissue capsule 12 can form between the living tissue 10 and blood vessels 11 and the implant 9, preventing substantial bio-integration.

An embodiment includes an implant framework material made of a biocompatible material such as a resin, e.g., silicone. The biocompatible material can include non-absorbable material. The resin, such as a silicone, can be a monomer that forms a polymer (e.g., a thermosetting plastic), oligomer, or a polymer having a reactive functional group. The resin can include a thermoset, thermoplastic, or a combination thereof Moreover, the implant can include a blend of polymers, copolymers, terpolymers, or combinations comprising at least one of the foregoing resins. The resin can also contain an oligomer, homopolymer, copolymer, block copolymer, alternating block copolymer, random polymer, random copolymer, random block copolymer, graft copolymer, star block copolymer, dendrimer, or the like, or a combination comprising at last one of the foregoing.

A “thermoset” solidifies when first heated and thereafter may not melt or mold without destroying the original characteristics. Thermosetting materials can include epoxides, phenolics, melamines, ureas, polyurethanes, polysiloxanes, polymers including a suitable crosslinkable functional moiety, or a combination comprising at least one of the foregoing.

A thermoplastic has a macromolecular structure that repeatedly softens when heated and hardens when cooled. Illustrative examples of thermoplastic polymeric materials include olefin-derived polymers, for example, polyethylene, polypropylene, and their copolymers; polymethylpentane-derived polymers, for example, polybutadiene, polyisoprene, and their copolymers; polymers of unsaturated carboxylic acids and their functional derivatives, for example, acrylic polymers such as poly (alkyl acrylates), poly (alkyl methacrylate), polyacrylamides, polyacrylonitrile, and polyacrylic acid; alkenylaromatic polymers, for example polystyrene, poly-alpha-methylstyrene, polyvinyltoluene, and rubber-modified polystyrenes; polyamides, for example, nylon-6, nylon-66, nylon-11, and nylon-12; polyesters, such as, poly(alkylene dicarboxylates), e.g., poly(ethylene terephthalate) (hereinafter sometimes designated “PET”), poly(1,4-butylene terephthalate) (hereinafter sometimes designated “PBT”), poly(trimethylene terephthalate) (hereinafter sometimes designated “PTT”), poly(ethylene naphthalate) (hereinafter sometimes designated “PEN”), poly(butylene naphthalate) (hereinafter sometimes designated “PBN”), poly(cyclohexanedimethanol terephthalate), poly(cyclohexanedimethanol-co-ethylene terephthalate) (hereinafter sometimes designated “PETG”), and poly(1,4-cyclohexanedimethyl-1,4-cyclohexanedicarboxylate) (hereinafter sometimes designated “PCCD”), and poly(alkylene arenedioates); polycarbonates; co-polycarbonates; co-polyestercarbonates; polysulfones; polyimides; polyarylene sulfides; polysulfide sulfones; and polyethers such as polyarylene ethers, polyphenylene ethers, polyethersulfones, polyetherimides, polyetherketones, polyetheretherketones; or blends or copolymers thereof.

The resin also can include other biocompatible resins such as, for example, a silicone, polysiloxane, poliglecaprone, polydioxanone, polyglactin, caprolactone, polyorthoester, polyethylene glycol, poly terephthalate, tyrosine, poly(ester amide), polyisobutylene, poly(ethylene terephthalate), polytetrafluoroethylene, polyurethane, polystyrene, polyamide, polyimide, bisphenol-alpha-glycidyl methacrylate, triethyleneglycol dimethacrylate, hydroxyethyl methacrylate, poly-p-chloroxylylene, phenolic resins, and the like. Phenolic resins can be obtained by condensation of phenol or substituted phenols with aldehydes. Suitable phenolic resins may include biocompatible phenol-aldehyde resins such as one-stage and two-stage phenol-formaldehyde resins, as well as polyvinyl phenol resins. Suitable one and two-stage phenol formaldehyde resins include resole and novolak resins. Examples of polyvinyl phenolic resins include o-hydroxystyrene, m-hydroxystyrene, p-hydroxy styrene, 2-(o-hydroxyphenyl)propylene, 2-(m-hydroxyphenyl)propylene, and 2-(p-hydroxyphenyl)propylene, and combinations, derivatives, or copolymers thereof

For the implant framework material, other biocompatible materials may be used, such as polytetrafluoroethylene (PTFE, available under the trade mark Teflon), polyethylene, polypropylene, nylon, calcium, coral, acellular bone, calcium hydroxyl apatite, and the like.

FIG. 5 shows a surface-textured implant 9, and FIG. 6 shows the implant 9 after being implanted in a living body. Living tissue 10 and blood vessels 11 grow near the surface of the implant 9; however, a scar tissue capsule 12 forms between the living tissue 10 and blood vessels 11 and the implant 9, preventing substantial bio-integration, similar to the implant shown in FIG. 4. Since the surface of this implant has a textured region 13, the implant may be better attached (physically) to the living tissue 10 and blood vessels 11 within the living body, relative to the implant 9 without surface texturing.

According to an embodiment, the biologically-active matrix material can include hyaluronic acid, collagen, and polylactic acid, acellular dermal matrix, protein, amino acid, carbohydrate, polyethylene terephthalate, polycarbonate, polylactic glycolic acid, glycolide, lactide, trimethylene carbonate, or a combination comprising at least one of the foregoing. Exemplary biologically active matrix materials include citrate esters (e.g., acetyl tri-n-butyl citrate, triethyl citrate, and the like), maleic acid esters, adipic acid esters, homopolymers and copolymers of L-lactic acid, D-lactic acid, D,L-lactic acid, glycolic acid, ε-caprolactone, N-methylpyrrolidone, trimethylene carbonate, p-dioxanone, 1,5-dioxepan-2-one, hydroxybutanoic acid, hydroxyvaleric acid, acid anhydrides (sebacic acid anhydride, maleic acid anhydride, dioleic acid anhydride, etc.), amino acids (L-amino acids, D-amino acids, mixtures of L- and D-amino acids) such as glycine, alanine, phenylalanine, tyrosine, asparagine, glutamine, aspartic acid, glutamic acid, ricin, hydroxylysine, arginine, valine, leucine, isoleucine, serine, threonine, cysteine, methionine, tryptophan, histidine, proline, hydroxyproline, etc., and the like; mixtures of such polymers, polyesters, polycarbonates, polyacrylic acids such as poly(α-cyanoacrylate), polyphosphates, amino acid polymers, polyacid anhydrides, proteins (gelatin, collagen, etc.), polyglycosides (chitin, chitosan, starch, etc.), and the like.

The biologically active-matrix can include such commercially-available products as Gore® BioA®, LifeCell™ Alloderm®, Bard™ Allomax™, and LifeCell™ Stratus®. As mentioned, the biologically-active matrix material can also be collagen containing tissue such as acellular dermis obtained from human, porcine, or bovine skin. In an embodiment, the biologically-active matrix material can also be a synthetic material such as Gore® Bio-A®, Ethicon Vicryl®, Coviden Dexon®, polyglycolic acid, Novus TIGR® Matrix, trimethylene carbonate, and the like.

The implant can also include an additive. The additive can be a pharmaceutical agent. Exemplary pharmaceutical agents include a bone growth factor, tissue growth factor, tissue-derived substance (e.g., albumin, globulin, chondroitin sulfate, fibronectin, fibrinogen or elastin), antibiotic (e.g., tetracyclines such as minocycline and doxycycline, macrolides such as clarithromycin or azithromycin, quinolones such as levofloxacin, or ketolides such as telithromycin), anti-inflammatory agent (e.g., non-steroidal anti-inflammatory drugs such as flurbiprofen, and steroids such as dexamethasone), naturally derived substance (e.g., azulene), bone-absorption inhibitor (e.g., bisphosphonate), inorganic compound (e.g., calcium phosphate, tricalcium phosphate, tetracalcium phosphate, hydroxyapatite), biological (e.g., platelet-rich plasma (PRP), fibroblasts, stem cells), and the like. The additive can be disposed in or on a surface of the implant. In an embodiment, the additive is dispersed in the resin or arranged on the biologically-active material.

FIG. 7 shows a surface-textured implant 9 including granules 14 of biologically-active matrix material, and FIG. 8 shows the implant 9 after being implanted in a living body and bio-integration occurs. As living tissue 10 and blood vessels 11 grow near the surface of the implant 9, blood vessels 11 grow into the granules 14. When blood vessels 11 grow into the granules 14, the granules may be consumed entirely by the blood vessels 11, allowing living tissue 10 and blood vessels 11 to occupy the space formerly occupied by the granules 14 (the biologically-active matrix material is therefore referred to herein as “bio-integratable”). Thus, bio-integration of the implant 9 is increased relative to the implants shown in FIG. 4 and FIG. 6. However, as shown in FIG. 8, since the granules 14 are only disposed in the implant 9 near the surface thereof and have a relatively small depth into the implant 9, bio-integration may be limited to the depth that the granules 14 penetrate into the implant 9 and become absorbed.

FIG. 9 shows a surface-textured implant 9 including granules 14 of biologically-active matrix material, and FIG. 10 shows that implant 9 after being implanted in a living body and bio-integration occurs. Similar to the implant shown in FIG. 7, as living tissue 10 and blood vessels 11 grow near the surface of the implant 9, blood vessels 11 grow into the granules 14. Thus, bio-integration of the implant 9 having biologically-active matrix material is increased relative to the implants shown in FIG. 4 and FIG. 6. Since the granules 14 are disposed throughout the implant 9 (with some in contact with each other), as FIG. 10 shows, blood vessels 11 and living tissue 10 can grow further into the implant than as shown in FIG. 8. However, since the biologically-active matrix material shown in FIG. 7 through FIG. 10 is in granule form, ingrowth of blood vessels 11 and living tissue 10 can be uneven or incomplete, resulting in inadequate bio-integration.

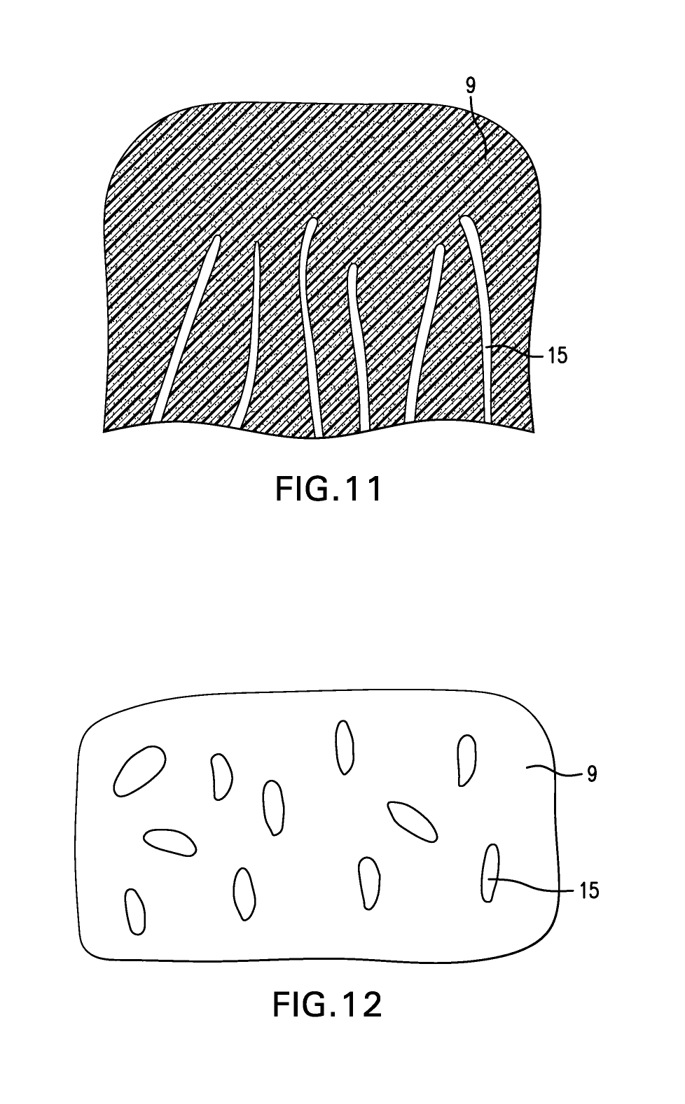

FIG. 11 shows a cross-sectional view of an implant 9 with strands 15 of biologically-active matrix material disposed therein according to an exemplary embodiment. Each strand 15 of biologically-active matrix material extends along an extending direction into the implant 9 in order to allow sufficient bio-integration with the living body into which the implant 9 is implanted. As the depth of the biological strand 15 is longer than the opening diameter thereof, blood vessels can grow a certain distance into the strand 15. Without being bound by theory, it is believed that the blood vessels can support biologically-active cells for a short distance after termination of the blood vessels. Sufficient tissue fluid will diffuse to support transitional cells. As tissue fluid decreases, the cells are no longer able to survive. The biological material is intimately connected to the synthetic material, (as in FIG. 26) i.e., the biologically-active material and resin, respectively. No capsule is able to form in this structure. As shown in FIG. 11, the length of the strands 15 along the extending direction can be greater than the width of the strands 15. The strands 15 of biologically-active matrix material can be arranged in any fashion, and in an embodiment the strands 15 extend into the implant 9.

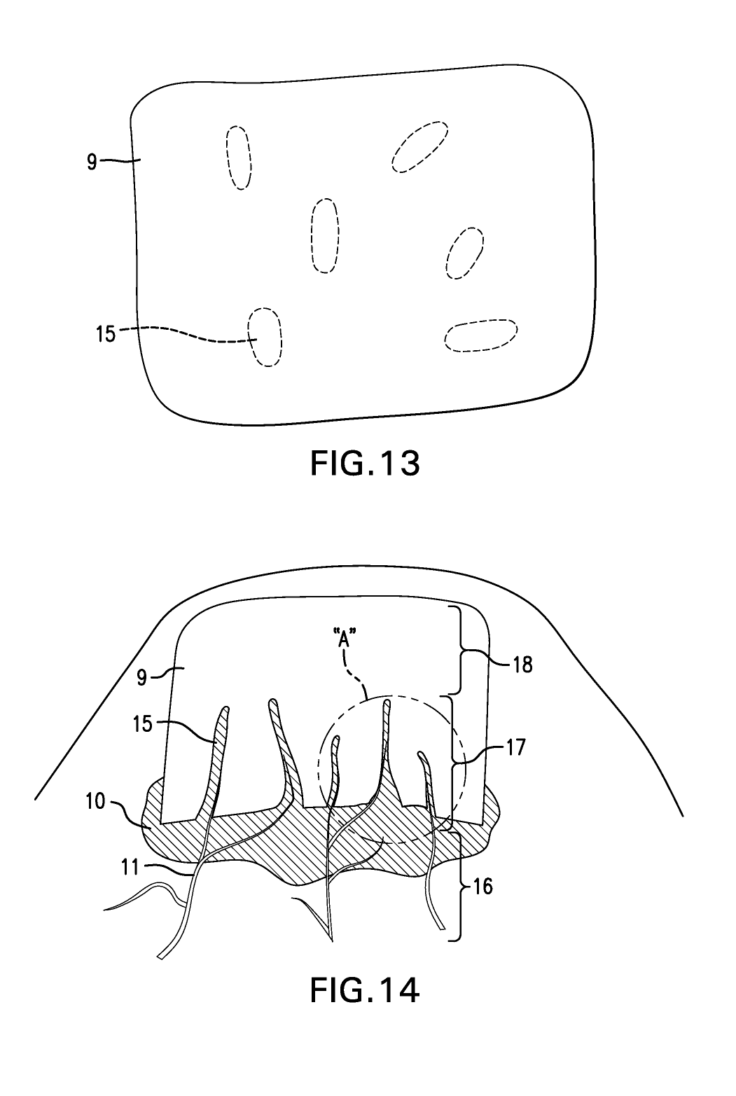

FIG. 12 shows a bottom view of the implant 9 shown in FIG. 11. Each strand 15 of biologically-active matrix material according to the present exemplary embodiment has as oval or rectangular shape but may have any shape. Each strand 15 of biologically-active matrix material is attached to and surrounded by the biocompatible material, e.g., silicone. Because each strand 15 is integrated with and surrounded by the biocompatible material, the structural integrity of the implant 9 can be improved. Alternatively, strands 15 of biologically-active matrix material can cross each other within the implant 9 to form a mesh. In this case, the strands 15 of biologically-active matrix material can contact each other in the mesh. FIG. 13 shows a top view of the implant 9 shown in FIG. 11. As indicated by the dotted outline of each strand 15 of biologically-active matrix material, the strands 15 do not penetrate the upper surface of the implant 9.

There are any number of alternative arrangements of the strands 15 in the implant 9 besides that shown in the present exemplary embodiment. For example, the strands can be arranged side to side, top to bottom, side to top, side to bottom, etc., or any combination of these arrangements. The strands 15 may not be straight but can have a curved shape. Further, strands 15 can be grown in by blood vessels 11 and living tissue 10 from more than one side of the implant 9 in certain situations where this may be indicated, e.g., joints.

FIG. 14 shows a cross-sectional view of the implant 9 in accordance with the present exemplary embodiment. The implant 9 is embedded within a living body, and bio-integration has occurred. As seen in FIG. 14, the implant 9 is surrounded by living tissue 10 and blood vessels 11. Scar tissue (not shown) can form between the living body and the implant 9 on the sides of the implant 9 exposed to the living body. Bio-integration is shown by blood vessels 11 and living tissue 10 penetrating into the implant 9 via the strands 15 of biologically-active matrix material. The blood vessels 11 and living tissue 10 that have penetrated into the implant 9 are referred to as “secondary” blood vessels and “secondary” living tissue. Secondary blood vessels 11 and secondary living tissue 10 grow into, partially dissolve, and absorb the strands 15 of biologically-active matrix material. Thus, the living body remodels the biologically-active matrix material, forming a transitional zone.

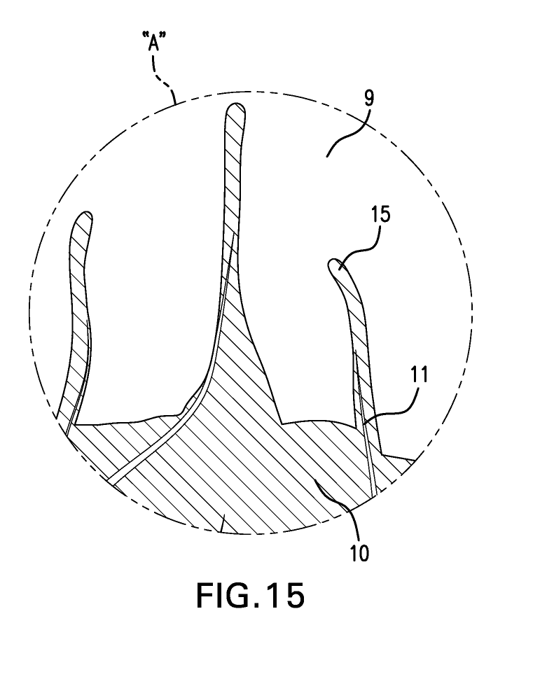

The process of bio-integration creates three connected regions in the area of the living body where the implant 9 has been implanted. The first region is a living zone 16, which is in the area of the living body where the blood vessels 11 and living tissue 10 grow originally. Next is the transitional zone 17, which contains secondary blood vessels 11 and secondary living tissue 10 that has absorbed the biologically-active matrix material strands 15 and therefore extends into the implant 9. The biologically-active matrix material strands 15 are at least partially bio-integrated in the transitional zone 17. The transitional zone 17 is shown in greater detail in FIG. 15. The last region is the non-living zone 18, which either is solely made of the implant 9 or may contain some part of the biologically-active matrix material strands 15 that have not been bio-integrated.

The three regions in the area of the living body where the implant 9 has been implanted create a junctional structure. As the secondary blood vessels 11 and secondary living tissue 10 penetrate further into the implant 9, they can become smaller and less able to penetrate. However, since the strands 15 of biologically-active matrix material extend into the implant 9 a certain distance, the implant 9 according to the present exemplary embodiment exhibits improved adhesion, strength, and durability once bio-integration has occurred. A scaffold created by the strands 15 due to the junctional structure holds the implant 9 to the blood vessels 11 and the living tissue 10.

By creating a transitional zone, a scar tissue capsule may not form between the implant 9 and the blood vessels 11 and the living tissue 10, and exteriorization of the implant can be facilitated. Thus, dental implants and fixation devices for external prostheses such as ears, noses, and the like can be more easily formed compared with other implants. The transitional implant can also find application in buried prostheses such as joint, facial, chin, and skull implants. In these implants, fixation to the transitional zone can prevent bone resorption commonly seen with conventional implants.

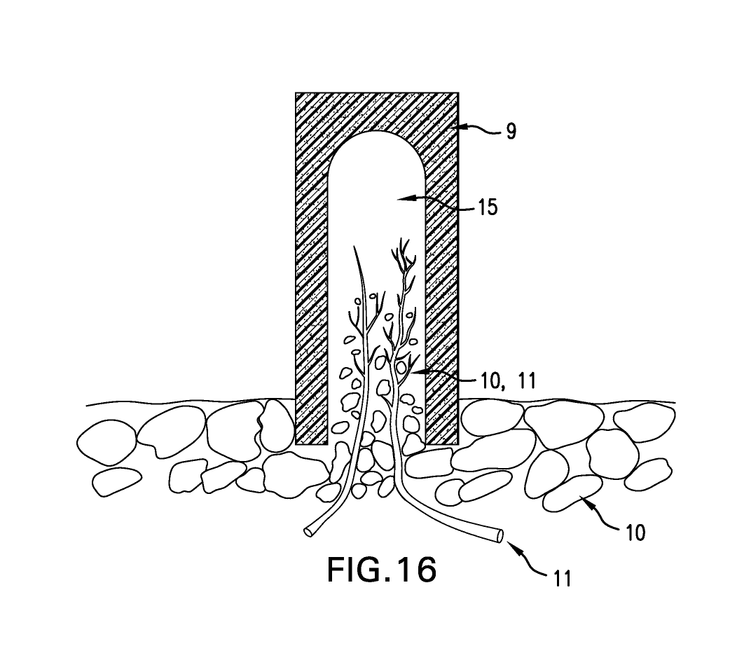

FIG. 16 shows a sectional view of an implant 9 with a biologically-active matrix material according to an exemplary embodiment. Similarly to the exemplary embodiment described above with respect to FIG. 11 through FIG. 15, the implant 9 contains a strand 15 of biologically-active matrix material, such as collagen. In the present exemplary embodiment, however, the implant contains a single strand 15 rather than a plurality of strands 15 of biologically-active matrix material. The present exemplary embodiment has the same three regions as described above, with blood vessels 11 and living tissue 10 from the living zone 16 growing into the strands 15 of biologically-active matrix material in the transitional zone 17, and the top portion of the implant 9 being the non-living zone 18. FIG. 16 shows blood vessels 11 in the transitional zone 17 become smaller as they grow further into the strands 15 of biologically-active matrix material, finally stopping before the non-living zone 18. Further, the alternative arrangement of strands described above with respect to FIG. 11 through FIG. 13 can be used in the present exemplary embodiment.

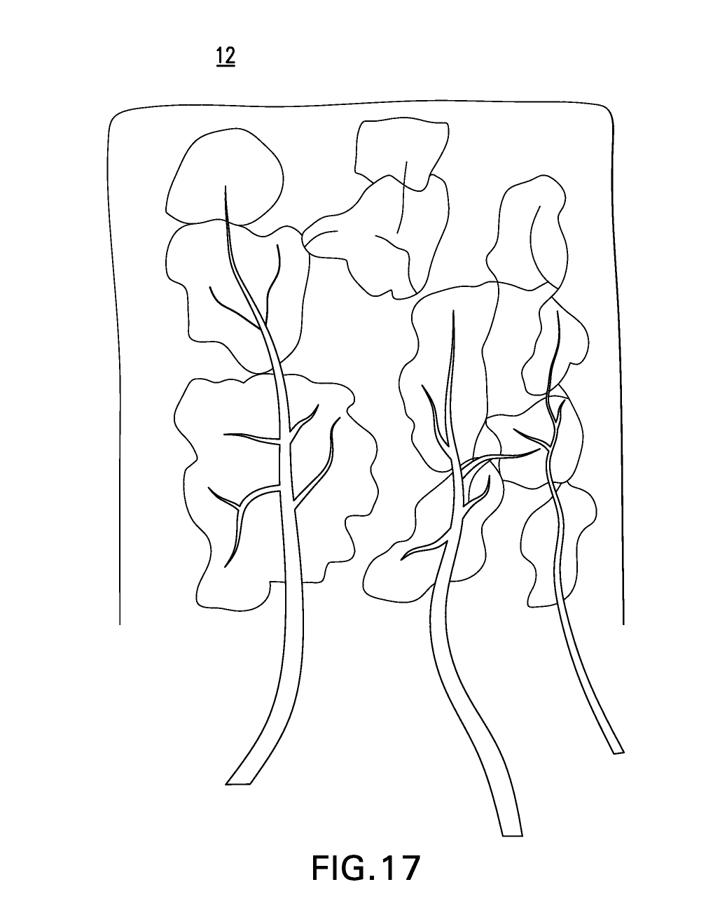

FIG. 17 shows a cross-sectional view of an implant 9 with granules 14 of biologically-active matrix material according to an exemplary embodiment. The present exemplary embodiment is similar to those shown in FIG. 11 through FIG. 15, except that instead of strands 15, granules 14 of biologically-active matrix material are used. The granules 14 are substantially in continuity in the present exemplary embodiment, so that the junctional structure can form, as described above. The granules 14 have a length that is about the same as a width thereof.

Exemplary embodiments, e.g., as shown in FIG. 16, show blood vessels 11 and living tissue 10 growing into the biologically-active matrix material from one side of the implant 9. This structure can be suitable for implants where a smooth and non-bio-integrated surface is desired. However, more thorough bio-integration can be possible if the biologically-active matrix material is accessible to blood vessels and living tissue from multiple sides of the implant. According to an embodiment, the length of biologically-active matrix material in the implant can be arranged such that the junctional structure is created, to form a scaffold between the resin and the blood vessels.

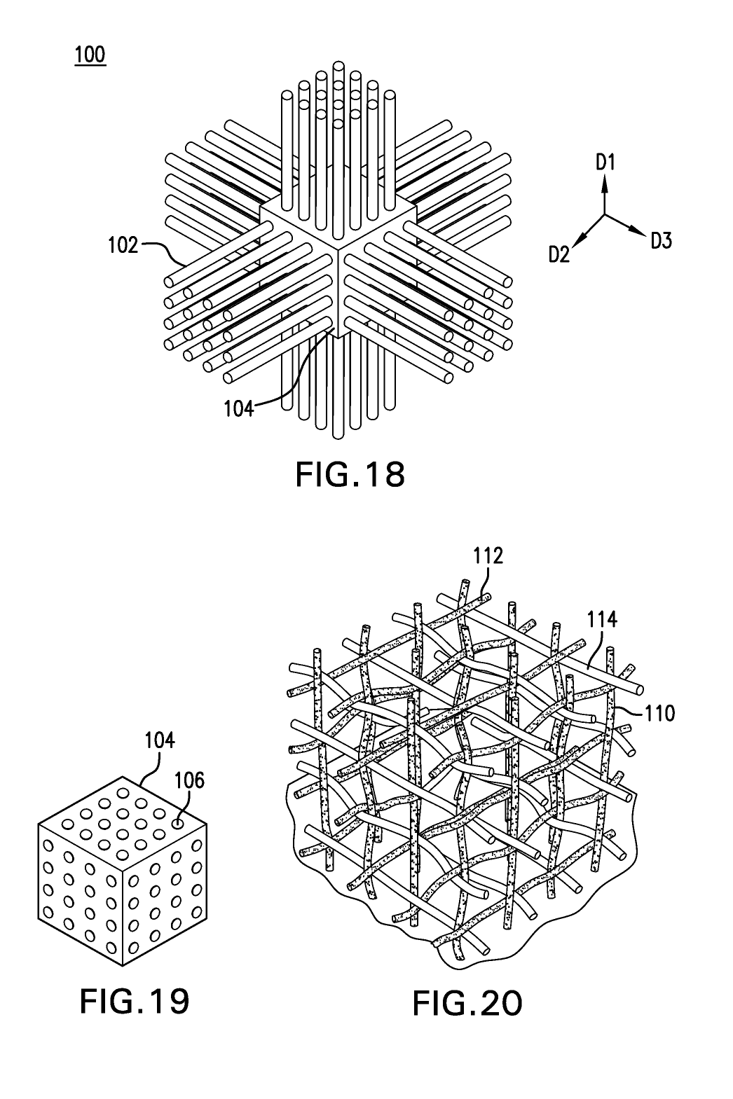

As shown in FIG. 18, a composite implant 100 has a biologically-active material 102 disposed in a resin 104. Individual members, e.g., strands, of the biologically-active material 102 can be arranged along any of three mutually orthogonal directions (first direction D1, second direction D2, or third direction D3) or at any angle with respect to the directions (D1, D2, D3). Thus the strands can cross perpendicular to one another or at any angle selected such as from 1° to 89° with respect to each other. After implantation tissue grows into the space having the biologically-active material 106 and surrounded by the resin 104, as shown in FIG. 19.

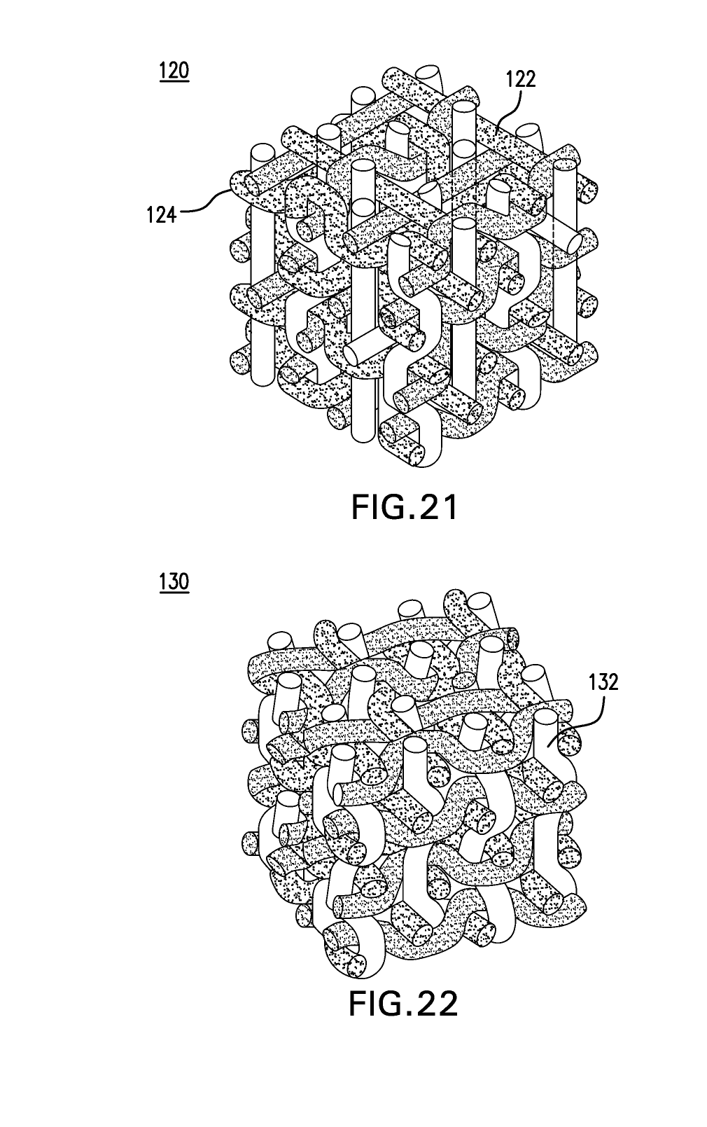

The biologically-active material can be rod, filament, thread-like, strands, tubules, and the like, or any combination thereof With reference to FIG. 20, a perspective view of biologically-active material can be arranged such that first tubule 112, second tubule 114, and third tubule 110 of biologically-active material cross one another. The tubules 112, 114, 116 of biologically-active material can have a hollow space or be substantially solid along its length, be porous in cross-section, or a combination thereof This arrangement of tubules 112, 114, 116 is shown without a covering of resin for convenience. FIGS. 21 and 22 respectively show partially woven 120 and completely woven 130 structures of biologically-active material. The partially woven structure 120 exhibits a partially interwoven strand 122 and can have a completely interwoven strand 124. The completely woven structure 130 contains completely interwoven strands 130. The partially interwoven strands 122 do not undulate around other strands as does the completely interwoven strands 130. Thus, in the structures shown in FIGS. 18-22, the composite implants has a three-dimensional array of biologically-active material. Strands of biological material can also be pre coated with resin (e.g., silicone) prior to weaving.

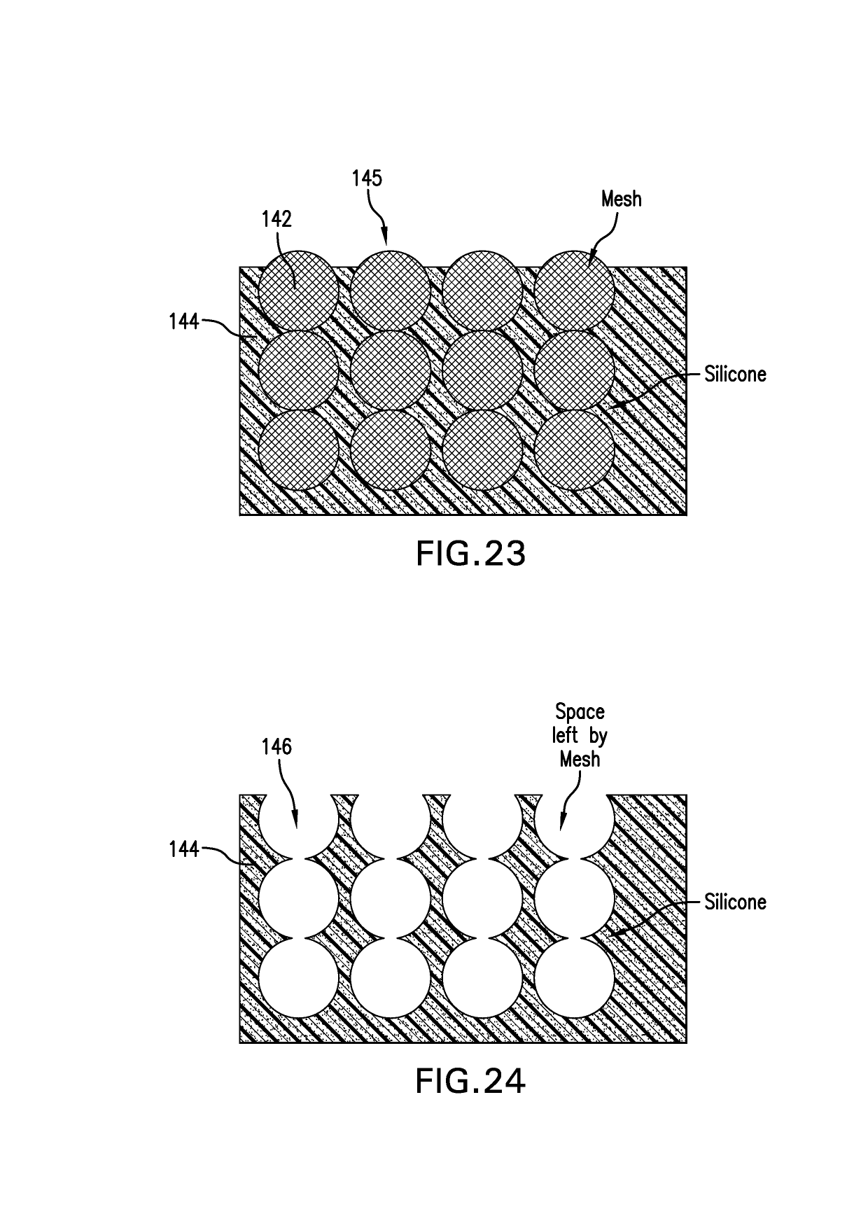

A cross-sectional view of a composite implant using the three-dimensional array of biologically active material 142 is shown in FIG. 23. Here, the individual strands are shown as circular in cross-section; however, they can have any cross-sectional shape, including ellipsoidal and the like. As depicted in the FIG. 23, adjacent strands of the biologically-active material 142 are in contact with one another. Resin 144 surrounds some of the biologically-active material with a surface 145 of the biologically-active material 142 being exposed without being covered by the resin 144. Due to the structural strength of the resin 144, the shape of the composite implant is maintained after partial absorption of the biologically-active material 142 and tissue growth into the composite implant. The resin 144 does not collapse even after partial absorption of the biologically-active material 142. However, resin 44 can be flexible. FIG. 24 shows the resin 144 having voids 146, which is a space that is occupied by biologically-active material 142 in the composite implant.

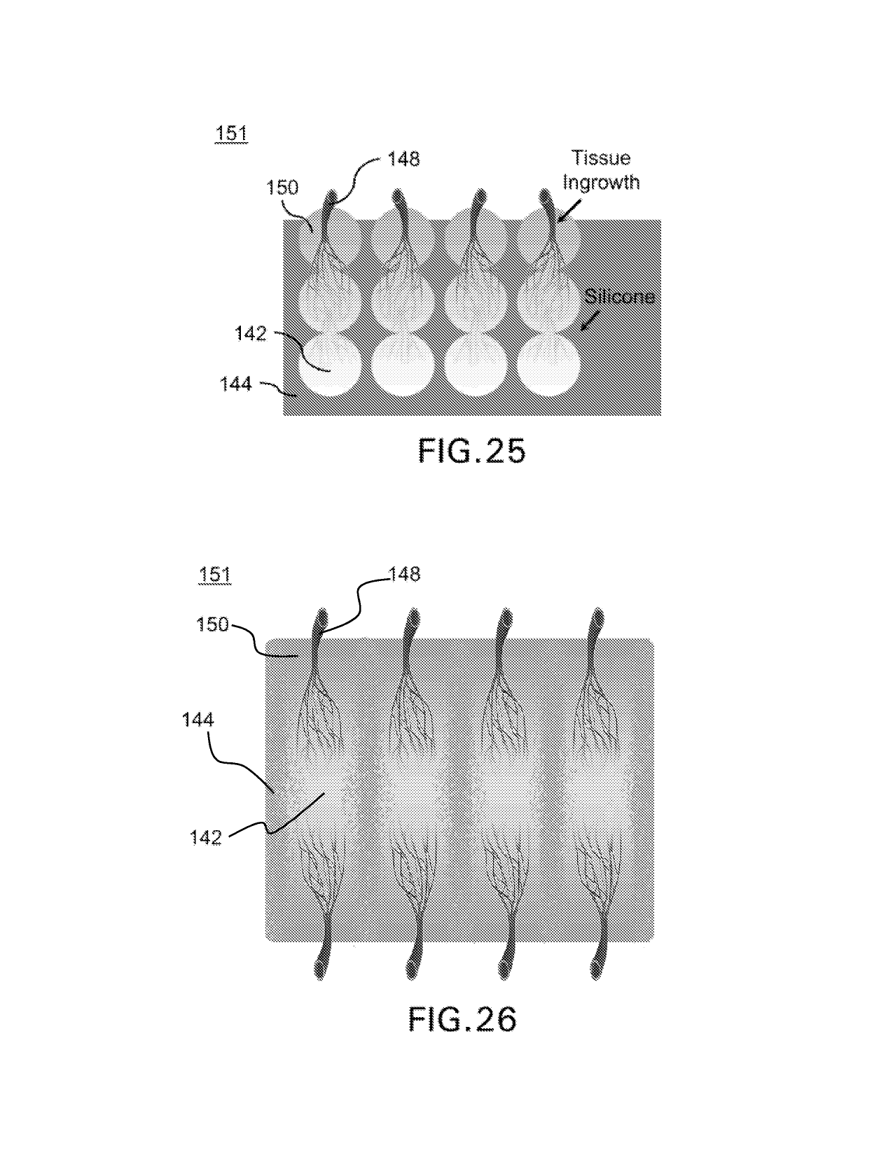

To illustrate the bio-integration of the composite implant 151, FIG. 25 shows a cross-section of the composite implant 151 after implantation of the implant into a subject. Here, the resin 144 covers the biologically-active material 142 such blood vessels 148 interface with and grow into the biologically-active material 142. Biological tissue 150 also grows into the biologically-active material 142, which is subject to partial absorption into the body of a subject having the composite implant 151. FIG. 26 shows a cross-sectional view of the composite implant 151 along a plane orthogonal to that of the cross-section shown in FIG. 25. In this view, blood vessels 148 and biological tissue 150 grow at opposing sites of the composite implant 151 and into the biologically-active material 142. Thus, the composite implant 151 is well-integrated into the host subject and can offer biomechanical stability to that portion of the host's anatomy.

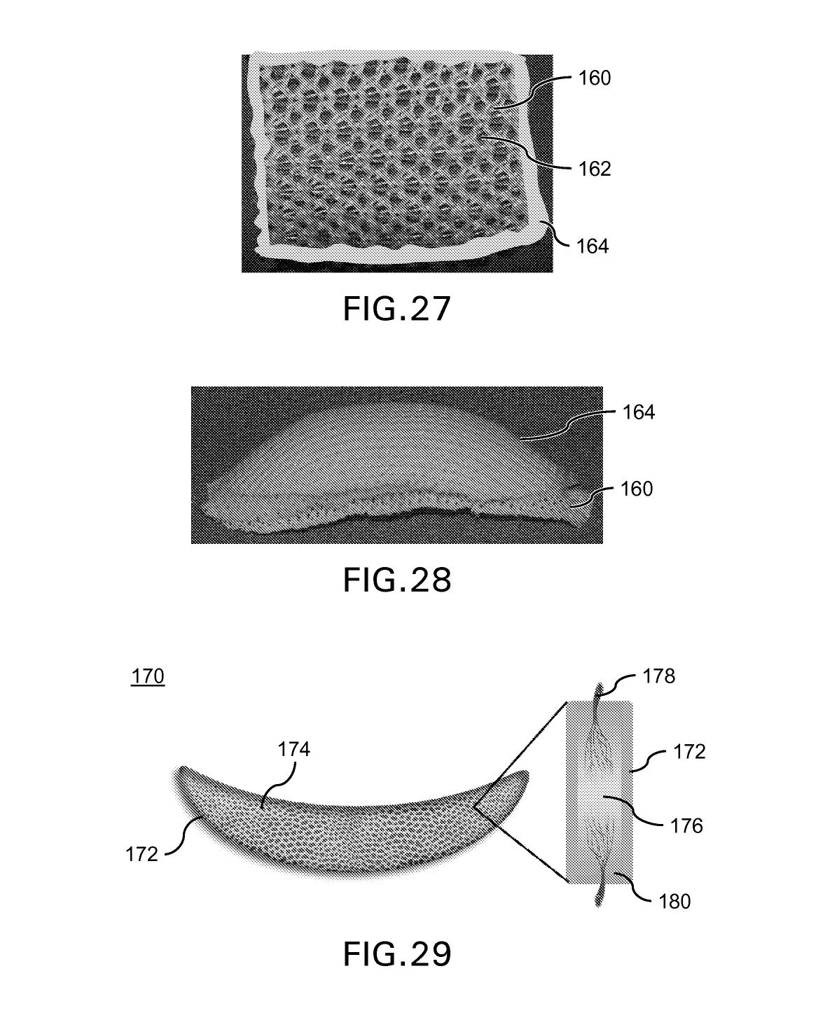

In an embodiment, a process for preparing a composite implant includes disposing a plurality of layers of biologically-active material as an array and contacting the plurality of layers with a resin. The resin is hardened to form the composite implant such that a channel in the mesh is configured to receive growth of the biological tissue and blood vessels, and the resin is biocompatible and non-absorbable. The biologically-active material can be coated with the resin and formed into a mesh prior to disposing the plurality of layers of biologically-active material as an array. A number of ways to dispose the resin on the biologically-active material can be used, including dipping the biologically-active material in the resin, spraying the resin on the biologically-active material, and the like. With reference to FIG. 27, the composite implant thus formed has a mesh pattern 160 with channels therethrough 162. The resin 164 can be disposed along an edge of the stack of the mesh. Alternatively, layers of the biologically-active material can be adhered by disposal of the resin between adjacent layers of the biologically-active material 160. The composite implant can be shaped into an anatomical shape as show in FIG. 28, which displays a chin implant 170. The curvature of the composite implant can be formed before cure of the resin. Alternatively, for certain resins, the composite implant can be heated above the glass transition temperature of the hardened resin and shaped to the anatomical shape with subsequent cooling below the glass transition temperature of the resin to arrest the composite implant in the anatomical shape. In another embodiment, the anatomical shape can be established by machining the composite implant by, e.g., compression molding, cutting (e.g., by using laser cutting, lathing, milling, drilling, and the like), and the like, or a combination thereof

FIG. 29 shows the chin implant 170 having channels 174 formed of biologically-active material 176 in a resin 172. Consequently, blood vessels 178 and biological tissue 180 can grow into the biologically-active material 176 upon implantation into a subject. As a result, the composite implant, e.g., the chin implant 170, has excellent vascularization, biological and physical stability, and lifetime when implanted.

While one or more embodiments have been shown and described, modifications and substitutions may be made thereto without departing from the spirit and scope of the invention. Accordingly, it is to be understood that the present invention has been described by way of illustrations and not limitation. Embodiments herein are can be used independently or can be combined.

All ranges disclosed herein are inclusive of the endpoints, and the endpoints are independently combinable with each other. The suffix “(s)” as used herein is intended to include both the singular and the plural of the term that it modifies, thereby including at least one of that term (e.g., the colorant(s) includes at least one colorant). “Optional” or “optionally” means that the subsequently described event or circumstance can or cannot occur, and that the description includes instances where the event occurs and instances where it does not. As used herein, “combination” is inclusive of blends, mixtures, alloys, reaction products, and the like. All references are incorporated herein by reference.

The use of the terms “a” and “an” and “the” and similar referents in the context of describing the invention (especially in the context of the following claims) are to be construed to cover both the singular and the plural, unless otherwise indicated herein or clearly contradicted by context. “Or” means “and/or.” It should further be noted that the terms “first,” “second,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity). The conjunction “or” is used to link objects of a list or alternatives and is not disjunctive, rather the elements can be used separately or can be combined together under appropriate circumstances.

It will be understood that when an element or layer is referred to as being “on” or “connected to” another element or layer, it can be directly on or directly connected to the other element or layer, or intervening elements or layers may be present. In contrast, when an element is referred to as being “directly on” or “directly connected to” another element or layer, there are no intervening elements or layers present.

What is claimed is:

1. A composite implant comprising:
a mesh comprising a biologically-active material configured to contact biological tissue and blood vessels;
a resin disposed on the mesh; and
a channel in the mesh which is configured to receive growth of the biological tissue and blood vessels,
wherein the resin is biocompatible and non-absorbable.

2. The composite implant of claim 1, wherein the mesh is a two-dimensional mesh, and a plurality of the two-dimensional meshes is arranged as stacked layers such that adjacent meshes are in contact with one another.

3. The composite implant of claim 2, wherein the resin is disposed between adjacent meshes.

4. The composite implant of claim 2, wherein the resin is disposed on an edge of the plurality of the two-dimensional meshes.

5. The composite implant of claim 1, wherein the mesh is a three-dimensional mesh.

6. The composite implant of claim 1, wherein the resin is disposed on a surface of the mesh.

7. The composite implant of claim 1, wherein the composite implant is functionally graded with the resin such that an amount of the resin varies along a dimension of the composite implant.

8. The composite implant of claim 1, wherein a portion of the biologically-active material is an exposed portion which is not covered by the resin.

9. The composite implant of claim 8, wherein the exposed portion is configured to receive the growth of blood vessels and biological tissue in response to being implanted in a subject.

10. The composite implant of claim 9, wherein the biologically-active material is configured to be a scaffold which is partially absorbed in response to being implanted in a subject with the resin being left in the subject.

11. The composite implant of claim 10, wherein the resin is configured to maintain the blood vessels and biological tissue after the scaffold is partially replaced with biological tissue.

12. The composite implant of claim 11, wherein the resin has a continuous structure.

13. The composite implant of claim 1, wherein the resin completely covers an outer surface of the absorbable material.

14. The composite implant of claim 1, wherein the composite implant comprises a shape of an anatomical feature.

15. The composite implant of claim 1, wherein the composite implant is a transitional implant comprising a transition region configured to transition from living biological tissue disposed in the resin to a portion without living biological tissue after implantation in a subject.

16. The composite implant of claim 1, wherein the biologically-active material comprises an acellular dermal matrix, collagen, protein, amino acid, carbohydrate, polyethylene terephthalate, polycarbonate, polylactic glycolic acid, glycolide, lactide, trimethylene carbonate, or a combination comprising at least one of the foregoing.

17. The composite implant of claim 1, wherein the resin comprises a silicone, epoxide, phenolic, melamine, urea, polyethylene, acrylic polymer, nylon, polypropylene, poliglecaprone, polydioxanone, caprolactone, polyorthoester, polyethylene glycol, poly terephthalate, tyrosine, poly(ester amide), polyisobutylene, poly(ethylene terephthalate), polytetrafluoroethylene, polyurethane, polystyrene, polyamide, polyimide, or a combination comprising at least one of the foregoing.

18. A process for preparing a composite implant, the process comprising:
disposing a plurality of layers of biologically-active material as an array;
contacting the plurality of layers with a resin; and
hardening the resin to form the composite implant,
wherein a channel in the mesh is configured to receive growth of the biological tissue and blood vessels, and the resin is biocompatible and non-absorbable.

19. The process of claim 18, further comprising:
coating the biologically active material with the resin; and
forming the biologically-active material, which is coated with the resin, into a mesh prior to disposing the plurality of layers of biologically-active material as an array.

20. The process of claim 18, further comprising shaping the composite implant into an anatomical shape.

21. A process of using a composite implant, the process comprising:
implanting the composite implant into a subject,
wherein the implant comprises:
a mesh comprising a biologically-active material configured to contact biological tissue and blood vessels;
a resin disposed on the mesh; and
a channel in the mesh which is configured to receive growth of the biological tissue and blood vessels,
wherein the resin is biocompatible and non-absorbable.
